(19) United States
(12) Patent Application Publication
(10) Pub. No.: US 2005/0055035 A1
(43) Pub. Date: Mar. 10, 2005

(54) Image-based stereotactic frame for non-human animals

(76) Inventors: Eric Richard Cosman JR., Belmont, MA (US); Eric Richard Cosman SR., Belmont, MA (US)

Correspondence Address: STEPTOE & JOHNSON LLP;Attn: Docket Administrator - Box USPTO, 1330 Connecticut Avenue, NW, Washington, DC, 20036, US

(21) Appl. No.: 10/851,664

(22) Filed: May 24, 2004

Related U.S. Application Data
(60) Provisional application No. 60/472,738, filed on May 23, 2003.

(51) Int. Cl. A61B 19/00

(52) U.S. Cl. 606/130

(57) ABSTRACT

A system and method can establish the stereotactic coordinates of anatomical targets in non-human subjects utilizing tomographic, volumetric, or projection imaging as for the purpose of doing anatomical and/or biological research. An imaging machine can produce data representative of anatomy or function in the body of the non-human subject. A mechanical reference frame can be fixed to the body of the non-human subject, and can have an associated stereotactic coordinate system. An index structure attachable or integrated with the mechanical reference frame can provide stereotactic index data in image data from the imaging machine. The stereotactic index data and the image data of the anatomy of the non-human subject can be used to develop the stereotactic coordinate positions of anatomical targets detected in the image data relative to the stereotactic coordinate system. Probe paths can be developed from desired directions to the stereotactic coordinate positions, and probe supports can guide probes along the probe paths. Various embodiments, computational techniques, and phantom bases can achieve desired research objectives.

### PRIORITY CLAIM

This application claims priority under 35 U.S.C. § 119(e) to U.S. Application No. 60/472,738, filed May 23, 2003, which is incorporated by reference in its entirety.

### TECHNICAL FIELD

This invention relates generally to advances in scientific systems and procedures for understanding the function and relative anatomy of non-human animals. More particularly, this invention relations to an improved method and system for the quantitative determination of positions targets, and the stereotactic positioning of probes at targets in the non-human animal anatomy, including the brain, based on MRI, CT, PET, and other imaging modalities.

### BACKGROUND

The field of human brain stereotaxy is advanced. Some stereotactic frames have been designed for use on non-human animals. Non-human stereotactic frames generally feature to brain target determination from landmarks or other markers on the skull.

### SUMMARY

In general, a bone-fixed, stereotactic, mechanical reference frame can be attached to non-human animal bony structures. A graphic reference structure attached to the mechanical reference frame can provide images of indicia located on the graphic reference structure and images of the non-human animal anatomy from MRI, CT, PET or other image scanners. Coordinates with respect to the stereotactic mechanical reference frame, of targets seen in the MRI, CT, PET or other types of images, can be determined using the images of the image indicia. Approaches to anatomical targets can be determined relative to the mechanical reference frame with probes. It is possible to determine coordinates with respect to the mechanical reference frame, of targets seen in the MRI, CT, PET or other types of image data, using the images of the image indicia. One can calculate target coordinates determined with respect to the mechanical reference frame using other image data and/or using other data sources, such as those which measure functional, neural-activity-related, atomic, or flow properties of anatomy, or those which use contrast-enhancing agents. In other examples, determination of target positions, using the graphic reference structure, that are derived from various or multiple imaging modalities, such as CT, x-ray, MRI, FMRI, DTI, MEC, EEG, phMRI, flow-sensitized MRI, and MRI with fiber-tract-tracing contrast agents, such as manganese, applied to an imaged animal subject can be achieved. In examples, the placement of probes and other instruments by a stereotactic guidance system attached to the stereotactic mechanical reference frame can be performed so that the probes are directed at calculated targets derived from the graphic reference structure. A probe can take many forms including, but not limited to, an electrode, a stimulation electrode, an ablative electrode, a recording electrode, an electrical measurement device, an electrical waveform generator, a bio-activity-monitoring device, a chemical-monitoring device, a chemical delivery device, a contrast-agent delivery device, a delivery device for neurochemical and/or genetic agents, a device for neurochemical and/or genetic monitoring, a needle, a needle configured for injection, a needle-like device, a device to be chronically implanted, and a beam of radiation.

In one aspect, a method of stereotactic target localization in the body of a non-human subject includes attaching a mechanical reference frame to the bony structures of the non-human subject, the mechanical reference frame being adapted to support at least one index element and to provide index data when the mechanical reference frame with the at least one index element imaged by an imaging machine to relate the position of the mechanical reference frame to image data from the imaging machine, imaging the body of the non-human subject and the mechanical reference frame with at least one index element, by the imaging machine to provide image data of anatomical positions in the body and to provide index data from the at least one index element, to provide stereotactic data that relates the positional relationship of the mechanical reference frame and the anatomical positions, and calculating the positional relationship of a target location in the anatomical positions relative to the mechanical reference frame using the index data and the image data.

The mechanical reference frame can be a head frame that is configured to be secured to the skull of a non-human animal by at least one attachment anchor. Attaching can include anchoring the at least one attachment anchor to the skull of the non-human animal. The method can include calculating a path to the target locations in relation to the mechanical reference frame based on the stereotactic data, or calculating the stereotactic coordinates associated with the image data in the stereotactic coordinate system, Calculating the path can include determining a path relative to the probe support so that a probe attached to the probe support can pass to a desired target location determined in the stereotactic data. The mechanical reference frame can include a probe support or an associated stereotactic coordinate system.

In another aspect, a stereotactic system for determining the coordinate position of an anatomical target in the body of non-human subject includes a mechanical reference frame configured to be rigidly attached to the bony anatomy of the non-human subject and to have an associated three-dimensional stereotactic coordinate system, and an index structure configured to attach to the mechanical reference frame having at least one index element that produces index data in image data when the index element is imaged by an imaging machine, so that when the imaging machine images the non-human subject with the mechanical reference frame and the index structure attached, the coordinate position of the anatomical target in the non-human subject can be determined in the stereotactic coordinate system from the target image data of the anatomical target in the image data.

The mechanical reference frame can include a head ring structure that is adapted to be rigidly attached to the skull of the non-human subject. The index element can include a tomographic index object that is detectable in at least one scan slice of the index structure by a tomographic imaging scanner to produce the index data. The tomographic index object can include a slice marker element which produces location data in the index data within a single tomographic image slice that define the three-dimensional coordinates of at least three non-collinear points in the stereotactic coordinate system when the index structure is attached to the mechanical reference frame. In examples, the tomographic index object can include an MRI index object that indexes data from imaging by an MRI image scanner. The slice marker element can include a diagonal element that is configured to be oriented non-parallel to the plane of the at least one image slice to produce the location data which can be used to determine the orientation of the plane of the tomographic image slice relative to the index structure. The diagonal element can include an MRI visible diagonal element that is detectable in the image data from an MRI scanner image. The system can include a probe support configured to attach to the mechanical reference frame and support a probe that is aimed at the coordinate position or a phantom base that enables developing a phantom target position on the phantom base at the coordinate position of the anatomical target, whereby the probe support can be attached to the phantom base and a probe path can be developed on the probe support so that when the probe support is attached to the mechanical reference frame, a probe can be passed to the anatomical target in the non-human animal.

Advantageously, an anatomical target determined or visualized in image scan data can be determined in the physical space of the non-human subject or in the three dimensional stereotactic coordinate system of the mechanical reference frame. Anatomical targets can be calculated with respect to the mechanical reference frame attached to the non-human subject, and the relative positions of anatomical targets and direction of probes or agents to anatomical targets can be determined. An advantage is that quantitative stereotactic study of anatomical targets can be achieved in, for example, neurophysiological study of the brain and brain function of animals. Another advantage is that electrical and chemical activity of targets seen in image data of animals can be determined by directing probes to the targets for the study of function in relation to the positional relationships of anatomical structures in animal organs such as the brain.

The methods and devices are directed at stereotactic application to non-human subjects which can include apes, monkeys, rats, mice, dogs, rabbits, cats, fish, frogs, pigs, and other creatures. It can also be applied to insects many of which have a firm shell or other structures which can be rigidly or semi-rigidly attached to with a mechanical reference frame.

The methods and devices can be directed at research into function and features of the anatomy of non-human subjects by stereotactic, quantitative localization of target positions in the subject's anatomy using application of a mechanical reference frame with image indicia. It also relates to use of imaging machines to produce image data of the anatomy and the image indicia from an imaging indexing element and/or localizer. In one example, the invention can be used for basic research in electrophysiological and neural science by animal experimentation.

Forms of the stereotactic system and method are disclosed herein in various embodiments. Specific embodiments of mechanical reference frames, image localizers, probe guides, probe guide blocks and guide chambers, phantom bases, arc systems, and computer and graphic displays are disclosed which are suited to the stereotactic system and its use.

### BRIEF DESCRIPTION OF THE DRAWINGS

In the drawings which constitute a part of the specification, embodiments exhibiting various forms and features hereof are set forth. Specifically:

FIG. 1 is a schematic diagram showing a stereotactic system for non-humans.

FIG. 2 is a schematic diagram showing a stereotactic probe carrier.

FIG. 3 is a schematic diagram showing a mechanical reference structure with a head ring.

FIG. 4 is a schematic diagram showing a stereotactic mechanical head ring with fixation posts.

FIG. 5 is a schematic diagram of a mechanical reference structure with imaging reference index structure attached.

FIG. 6 is a schematic diagram showing a head ring with indexing reference structure having rods and diagonals and x-ray indicia.

FIG. 7 is a schematic diagram showing a head ring with ear bar alignment and various skull-clamping positions.

FIG. 8 is a schematic diagram showing a reference structure attached to a skull with a variety of skull fixation post and rod positions.

FIG. 9 is a schematic diagram showing a reference structure attached to a skull with various skull fixation positions.

FIG. 10A is a schematic diagram showing a skull bone drill and guidance system.

FIG. 10B is a schematic diagram showing a skull screw and alignment system.

FIG. 10C is a schematic diagram showing a skull screw and pin alignment system.

FIG. 10D is a schematic diagram showing a skull anchor and skull screw with a pin fixation system.

FIG. 11 is a schematic diagram showing a pointed skull pin and drive.

FIG. 12 is a schematic diagram showing a stereotactic probe guide and arc system on a head ring and skull.

FIG. 13 is a schematic diagram showing a stereotactic arc system and head ring on a skull.

FIG. 14 is a schematic diagram showing a probe alignment system with a phantom base.

FIG. 15 is a schematic diagram showing multi-hole probe guide block attached to a skull and a image-indexing reference structure attached to the guide block.

FIG. 16 is a schematic diagram showing a sectional view through a guide block and attached indexing graphic reference structure with stereotactic indicia.

FIG. 17 is a schematic diagram, in sectional view, showing an mechanical reference frame attached to the non-human animal skull with a graphic imaging index reference structure and probe guide block.

FIG. 18A is a schematic diagram showing a head post with pointed skull screw.

FIG. 18B is a schematic diagram showing a head post with skin punch.

FIG. 18C is a schematic diagram showing a head post with skull drill.

FIG. 18D is a schematic diagram showing a head post with skull anchoring screws.

FIG. 19 is a flow diagram showing a process of stereotactic target determination on non-human subjects.

### DETAILED DESCRIPTION

Brain stereotaxy is described, for example, in portions of U.S. Pat. No. 4,608,977; System Using Computed Tomography as for Selected Body Treatments; Russell A. Brown, issued Sep. 2, 1986, and the book Tumor Stereotaxis by P. T. Kelly, W. B. Saunders Company, 1991, each of which is incorporated by reference in its entirety. Portions of the Kelly book describe some stereotactic frames that have been designed for use on non-human animals. Examples of non-human stereotactic frames are available from David Kopf Instruments, and they generally relates to brain target determination from bony landmarks on the skull. For example, a method for imaged-based stereotaxy in monkeys is presented in the paper by D. W. Risher, X. Zhang, E. Kostarczyk, A. P. Gokin, C. N. Honda, and G. J. Giesler, Jr. entitled “A method for improving the accuracy of stereotactic procedures in monkeys using implanted fiducial markers in CT scans that also serve as anchor points in a stereotactic frame”, published in the Journal of Neuroscience Methods, Volume 73, Pages 81-89, 1997, which is incorporated by reference in its entirety.

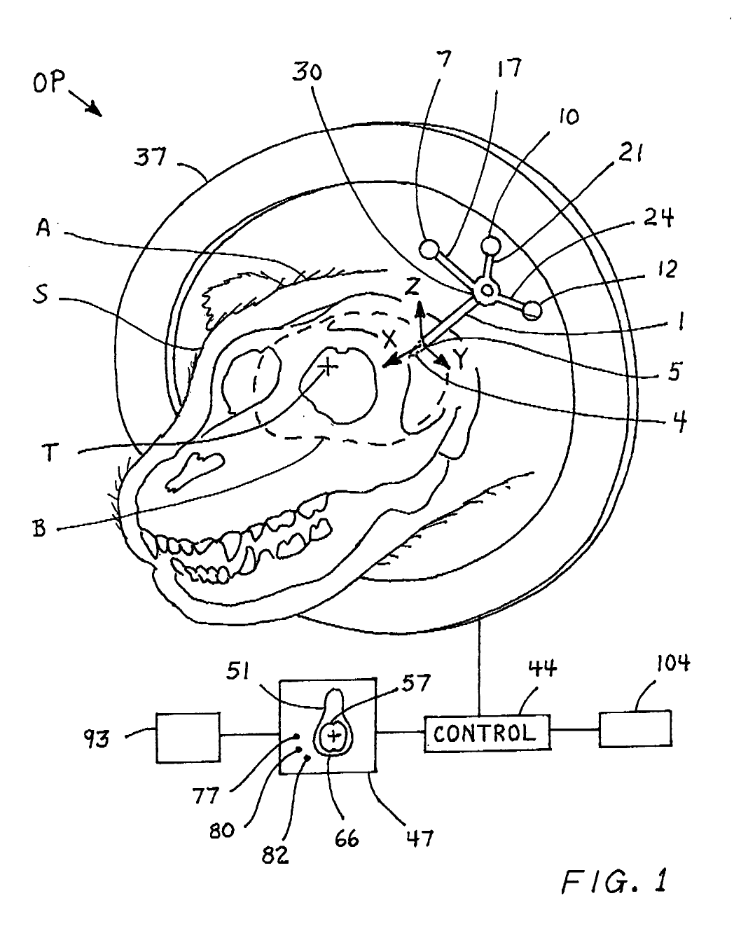

Referring to FIG. 1, in one embodiment, a stereotactic mechanical reference frame includes a mechanical post 1 is fixed directly to the skull S of a non-human animal A. The post 1 is rigidly affixed to the skull S by a screw 4 that is firmly screwed into the bone of the skull S. A stereotactic coordinate frame reference 5 associated with the stereotactic, mechanical reference frame is indicated schematically by the by the Cartesian coordinate axes X, Y, and Z with respect to post 1. A graphic or indexing reference structure comprising a set of mechanical index markers 7, 10, and 12 is attached to post 1 by rigid arms 17, 21, and 24, respectively, which can be arranged in a rigid body. The rigid body is attached in a fixed, predetermined position on post 1 by attachment screw 30. The index markers 7, 10, and 12 can include materials that are detectable positions when the graphic reference structure is imaged in an image scanning machine, such as CT, MRI, x-ray, or other kinds of imaging machines.

Also shown in FIG. 1 is an imaging machine 37 which can be, for example, a MRI, CT, PET or other tomographic, volumetric, or planar projective imaging system. The imager 37 produces an image of the anatomy of the non-human animal's skull S and tissue around and inside the skull S such as the brain indicated by the dashed contour B as well as an anatomical target within the brain such as target T. The imaging system can include a control element 44 which includes electronics, software, and data collection elements to produces image data related to the images from the scanner 37. The data from element 44 can produce graphic images of the anatomy of animal A as shown schematically by display element 47. The image of the skull S is shown as contour 51, the brain B as contour 57, the target T as position 66. The index markers on the graphic reference structure, such 7,10, and 12, are adapted to shown up on the image data from scanner 37 and control unit 44 as image indicia 77, 80, and 82, respectively. Other embodiments of the graphic reference structure can include more than three index markers to suit scientific purposes and/or the type of imaging data collected by image scanner 37 and control unit 44.

The data from control 44 related to the indicia 77, 80, and 82 can be inputted to, controlled by, or managed by computer, software elements, programs, or manual graphic computation element 93. Element 93 can be used to determine the coordinate position of the target image 66 with respect to the stereotactic mechanical reference frame 1 and the stereotactic coordinate frame 5 associated with the graphic reference structure. The target T can be an anatomical structure, a region or focus of neurological or neurochemical activity, which can be detected by image scanner 37 and associated with target image data related to location 66 on display element 47. The target indicated by position 66 can be imported into the control 44 and display 47 from another data source 104 such as another image scanner, MEG machine, or EEG system.

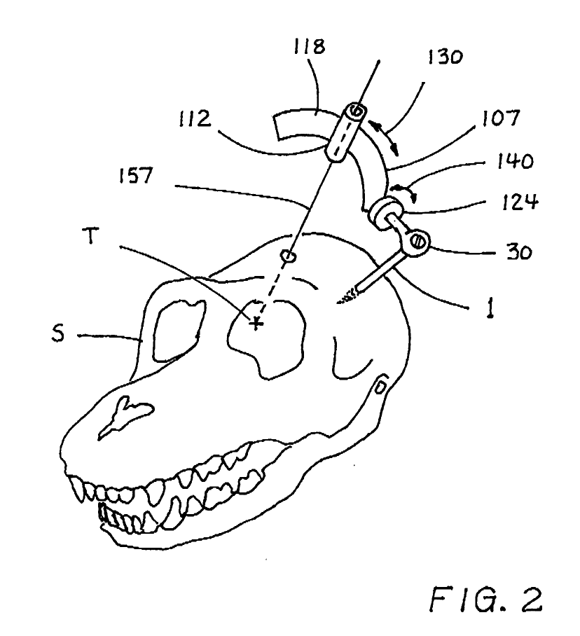

Referring to FIG. 2, a probe carrying system 107, which can also be referred to as an arc system or a stereotactic guidance system, is attached to the post 1, which is affixed to the skull S as shown in FIG. 1. Referring to FIG. 1, the rigid indicia structure and graphic reference structure including arms 17, 21 and 24, and markers 7, 10 and 12, has been removed by loosening screw 30, and replaced by probe carrying system 107 which is secured to post 5 by screw 30 in a predetermined orientation as shown schematically in FIG. 2. Referring again to FIG. 2, probe guide 112 can be moved on arc 118 over angular ranges indicated by arrow 130 and can be moved over another angular degree of freedom on trunion 124 over an angular range indicated by arrow 140. The probe carrier 112 can be set to direct probe 157 along a probe path direction to aim at target T as calculated from the image scan data as shown in FIG. 1. The probe carrying system can have translational and/or rotational degrees of freedom so that the position of any target T can be achieved by probe carrier from any direction. The degrees of freedom can have quantitative position scales to enable directing a probe, such as 157, accurately to a target coordinate position with respect to post 1 or with respect to the graphic reference structure of FIG. 1. In another embodiment, the probe carrier 107, indicia structure, and stereotactic mechanical reference frame 1, or any subsets of these elements, can be integrated into a single, unified apparatus which does not require the separation of these elements. In another embodiment, the stereotactic reference frame 1 can attach to more than one location on the skull S to reduce torques at the interface of the frame 1 and the skull S and to give the affixation greater mechanical stability.

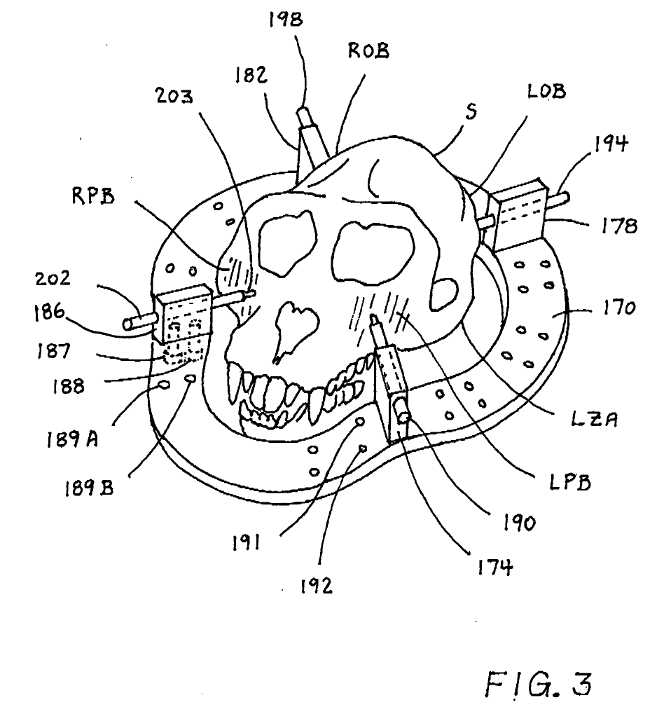

Referring to FIG. 3, an embodiment of a stereotactic mechanical reference frame can include a head ring 170 that can be a rigid structure which encircles the skull S of the non-human animal. The skull S is a schematic shown in FIG. 3 as the skull of a monkey, for example, the macaca mulatta. The skin of the non-human animal is not shown in FIG. 3 so that the attachment of head ring 170 to skull S can be simply illustrated. Posts 174, 178, 182, and 186 are rigidly attached to the head ring 170. Attachment rods 190, 194, 198, and 202 pass through posts 174, 178, 182, and 186, respectively, and attach to the skull S. The rods can be fixed in the posts to hold the head ring 170 firmly and rigidly to the skull S. In one embodiment, the posts can be unified with, or integrally fixed to the head ring. In another embodiment, each post can be independently adjusted, pre-adjusted and/or fixed in a variety of locations and orientations so that the direction of the attachment rods 190, 194, 198, and 202 can be configured to improve the mechanical stability and rigidity of the attachment of the head ring 170 to the skull S. This adjustability can be accomplished, for example, by drilling multiple holes in the head ring 170 such that each post can be attached to the head ring in a number of different positions. For example, in one embodiment of the system in FIG. 3, the post 186 can be positioned, oriented, or angulated by different attachment positions to head ring 170. Bolts 187 and 188 pass from below through holes drilled in head ring 170 and screw into the post 186. Multiple hole positions such as 189A and 189B in head ring 170 enable different attachment positions of post 186 to ring 170. Other holes, such as 191 and 192, enable the adjustment of post 174 and rod 190. Similar adjustment holes on ring 170 accommodate the other posts 178 and 182 orientation and position selection. One advantage of adjustable post and rod positions is that the attachment point of each rod, such as point 203 for rod 202, can be chosen to suit the particular shape of skull S, to suit the variations in thickness of the skull bone, and/or to suit other scientific needs.

Referring to FIG. 3, the attachment of each pair of post and attachment rod, 174 and 190, 178 and 194, 182 and 198, 186 and 202, can be configured to attach to the skull at the Left Periorbital Bone Region indicated by shaded region LPB, at the Left Occipital Bone LOB, at the Right Occipital Bone ROB, and at the Right Periorbital Bone Region indicated by shaded region RPB, respectively. The LPB comprises the region near the left ocular orbit of skull S, and can include the left orbital bone itself, the anterior portion of the left zygoma LZA, and the bone of the snout adjacent to the zygoma. The RPB comprises the region near the right ocular orbit of skull S, and can include the right orbital bone itself, the anterior portion of the right zygoma, and the bone of the snout adjacent to the zygoma. The LOB, ROB, LPB, and RPB contain bone which is typically among the heavier bone of the skull S of a monkey, such as the macaca mulatta. One advantage of attaching the head ring 170 by rods fixed to those regions of the skull S is that those regions can comprise heavier bone structures which are less fragile and provide a more stable attachment or anchoring point, thus reducing the chance of displacement of the ring 170 relative to S. Another advantage of the attachment of pin, rods, screws or other hardware to those regions of the skull S is that those regions typically contain bone which is substantially flat or which has small curvature; this quality can facilitate attachment to these locations and make attachment to these locations more stable. For instance a spike is less likely to slip, and a screw is better seated on a flat surface. The attachment rod 194 attached at the LOB and the attachment rod 202 attached at the RPB can be oriented to be on the opposite side of the skull S. This can have the advantage of stable mechanical attachment to the skull S by substantially clamping the skull S between the posts 178 and 186. Similar opposing clamping can be achieved between opposite posts 182 and 174 related to rods 198 and 174, respectively, which attach to the skull S at ROB and LPB, respectively.

In another embodiment, more than four posts and rod pairs can be used on head ring structure 170 to clamp or hold skull S to suit scientific needs. Other attachment points to skull S can be used. In another embodiment, only 3 post and rod pairs can be used on head ring 170 to stably fix head ring 170 to skull S. In another embodiment, only two post-and-attachment-rod pairs can be used on ring 170 to attach to skull S. In examples, the attachment rods can be sharpened, pointed rods that dig into the outer surface of skull S, or screws that can screw into skull S at the contact point, or pins that attach into pre-drilled socket holes in skull S, or forked or cusp-tipped rods that dig in and grip the bony skull.

In one embodiment, such as that in FIG. 3, the opposing attachment rods, such as 190 and 198, or 194 and 202, can be are substantially collinear or coplanar to reduce torques and give the affixation of the head ring 170 to the skull S mechanical stability. In another embodiment, the head ring is attached to skull S of a non-human animal, such that there is at least one attachment rod which attaches to the skull S in the LPB or the RPB, and at least one attachment rod which attaches to the skull S in the bone of the occipital region of the skull. For example, in one embodiment, a single post and rod can attach to ring 170 at the rear of skull S and attach near the midline position on the occipital bone.

Referring to FIG. 3, an embodiment of a stereotactic, mechanical reference frame, such as the head ring 170, is configured to the size and shape of the skull S of the non-human animal. For example, in one embodiment, the skull S is that of a monkey, such as the macaca mulatta, whose typical skulls are smaller than those of typical human beings. In one embodiment, the stereotactic mechanical reference frame, such as head ring 170, is configured to closely fit the skull of a monkey, such as the macaca mulatta. In another embodiment, the area of the volume surrounded by a stereotactic mechanical reference frame, such as head ring 170, its posts, such as 174, 178, 182 and 186, and its rods, such as 190, 194, 198, 202, is smaller than that which would practically accommodate the typical human head for the purpose of image-based stereotaxy. In another embodiment, the inner opening of head ring 170 spans no more than 6.3 inches in the direction configured to substantially align with the medial axis of skull S, and not more than 4.6 inches in the direction configured to substantially align with the right-left axis of the skull S. In another configuration, a stereotactic mechanical reference frame is configured so that each attachment rod spans no more than 1 inch between its point of contact at skull S and its point of contact at its stereotactic mechanical reference frame, when the stereotactic mechanical reference frame is attached to skull S of a non-human animal. For example, when head ring 170 is sized so that when it is attached to the skull S of a non-human animal, such as a monkey, such as macaca mulatta, the distance along each rod, from its respective attachment point to its respective post, is not more than 1 inch. For example, head ring 10 is sized so that when it is attached to skull S, the distance between attachment point 203 and post 186 along rod 202 is not more than 1 inch. One advantage of this configuration to the skull size and shape of the non-human animal is an improvement in the mechanical stability of attachment between skull S and the stereotactic mechanical reference frame, such as head ring 170.

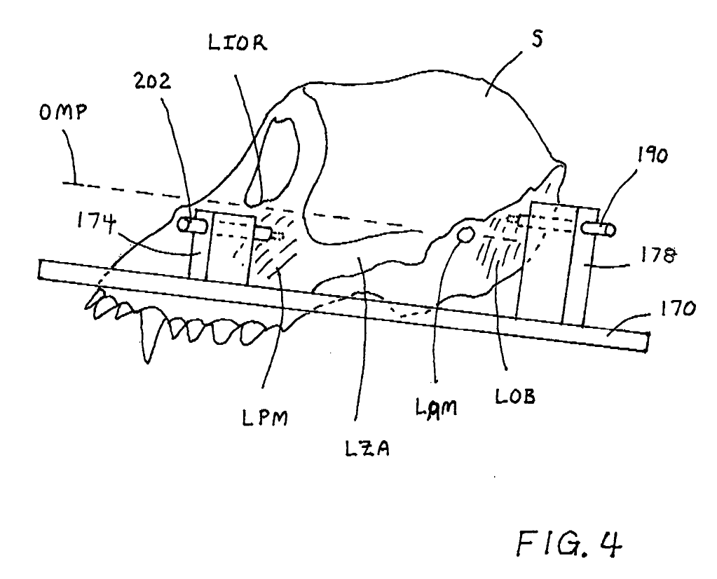

Referring to FIG. 4, a head ring 170, as described in connection with FIG. 3, is attached to the skull S of a non-human animal. The post 178 is longer than the post 174 so that the head ring 170 is substantially parallel to the orbitomeatal plane indicated by the dashed line labeled OMP. Pins 190 and 202 sercure the posts to the skull. The OMP can be defined as passing through any three of the following points: the left auditory meatus LAM, the left occipital bone LOB, and the left infraorbital ridge LIOR. The OMP is typically substantially parallel to the AC-PC line, which passes through the anterior commissure and the posterior commissure in the brain of many non-human animals, and defines one of the principle directions of some animal brain atlases. The posts 174, 186, 178, and 182 shown in FIG. 3 are also configured such that their respective rods have direction which is substantially perpendicular to their respective attachment points in the LOB, ROB, LPB, and RPB. One advantage of this configuration is that the attachment rods can be less likely to slip than they would if they approached the surface of the skull S at more glancing angles.

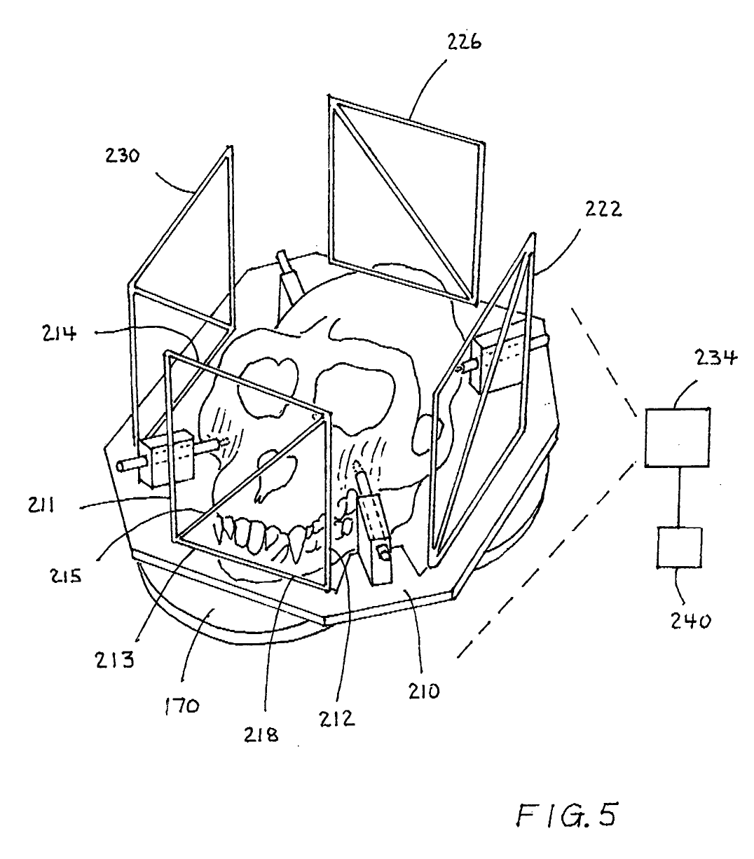

Referring to FIG. 5, a graphic reference structure or image index structure is attached to the head ring 170 in a predetermined position. The reference structure includes a bottom plate 210 that can be fixed in a known position to ring 170 by securing members, such as screws or clamps, so that the graphic reference structure can be attached and removed from ring 170. Structures 218, 222, 226, and 230 are configured to produce image indicia data when scanned with an image scanner represented schematically by element 234, so that the coordinates of imaged targets can be determined relative to the head ring 170.

The structures 218, 222, 226 and 230 can, in one embodiment, include a geometric array of rods or channels arranged on the sides of a rectangle, and diagonal rods/tubes arranged on diagonals or in an oblique configuration. For example, structure 218 can have vertical parallel rods 211 and 212, horizontal parallel rods 213 and 214, and at least one diagonal rod 215. The rods can be constructed with materials that are detectable in CT, MRI, PET or other imaging machines. A planar image slice through structure 218 can produce index images or image indicia in the image scan data for that slice corresponding to the intersection of the image slice plane at the parallel and diagonal rods. The image indicia detected for each of the structures 218, 222, 226, and 230 can produce sufficient image data to calculate the position of the image slice with respect to the image reference structure 218, 222, 226 and 230 and thus to the head ring 170, to which the image reference structure is attached in a known location. In this way, the coordinates of anatomical points or structures detected in the image data and in the scan slice can be determined relative to the structures 218, 222, 226 and 230 an relative to the head ring 170. Examples of image reference structures and head rings used in human stereotaxy to determine stereotactic coordinate positions of anatomical targets is illustrated by the CRW stereotactic system produced by Radionics, Inc. of Burlington, Mass., which is described in portions of the book entitled “Handbook of Stereotaxy Using the CRW Apparatus”, edited by D. Thomas and M. Pell, Williams and Wilkins, Co., 1994, which is incorporated herein by reference in its entirety.

Also shown in FIG. 5 is image scanner 234, which can be MRI, CT, PET, x-ray or other tomographic, volumetric, or planar imaging system. The image scanner is associated with element 240 can include controls, electronics, graphic display, computational systems, manual computation, mechanisms to associate scan data from 234 with other scan data, and other systems and methods related to collecting image data, producing scan images, and computing the coordinates of targets relative to the head ring 170.

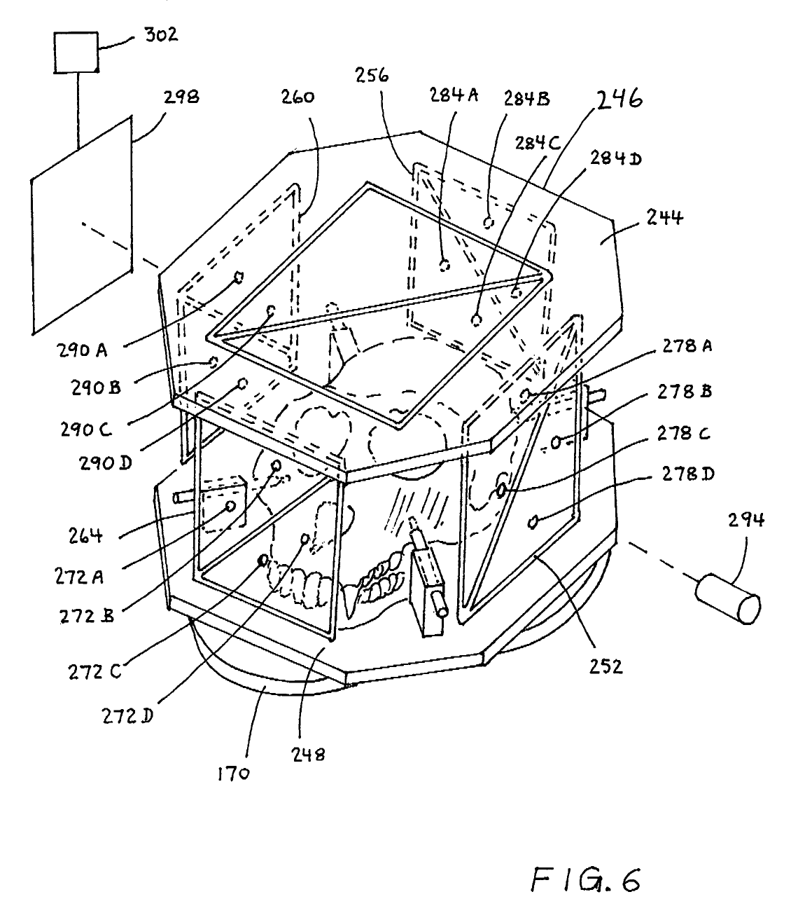

Referring to FIG. 6, one embodiment of an index reference structure 246 is attached to the head ring 170 in a known position. For example, the index reference structure 246 can be similar to that described above. The index reference structure 246 can include a bottom plate 248, a top plate 244, and parallel plus diagonal rod structures 252, 256, 260, 264 and 268 which are configured to produce image indicia data when scanned with an image scanner like that shown in FIG. 5. Image indicia data produced by structures 252, 256, 260, 264 and 268 in sagittal, coronal and axial planar image scans can be used to determine the coordinates of imaged targets. The index reference structure in FIG. 6 also includes reference elements 272A, 272B, 272C and 272D; 278A, 278B, 278C and 278D; 284A, 284B, 284C and 284D; and 290A, 290B, 290C and 209D which can produce image indicia when scanned by a projective x-ray apparatus, represented schematically by an x-ray emitter 294, detector 298, and control element 302. The indicia are configured so that the coordinates of anatomy and implanted objects in and around the skull S which are visible when scanned one or multiple times by x-ray imaging scanner comprising 294, 298, and 302. This system and method can be used, for instance, to confirm the location of electrodes, probes, needles, or other objects that can be imaged by an x-ray scanner, which have been implanted in and about the skull S of a non-human animal. Examples of the use of x-ray visible structures with index markers such as those shown in the embodiment in FIG. 6 can be found in the CRW Handbook reference described above.

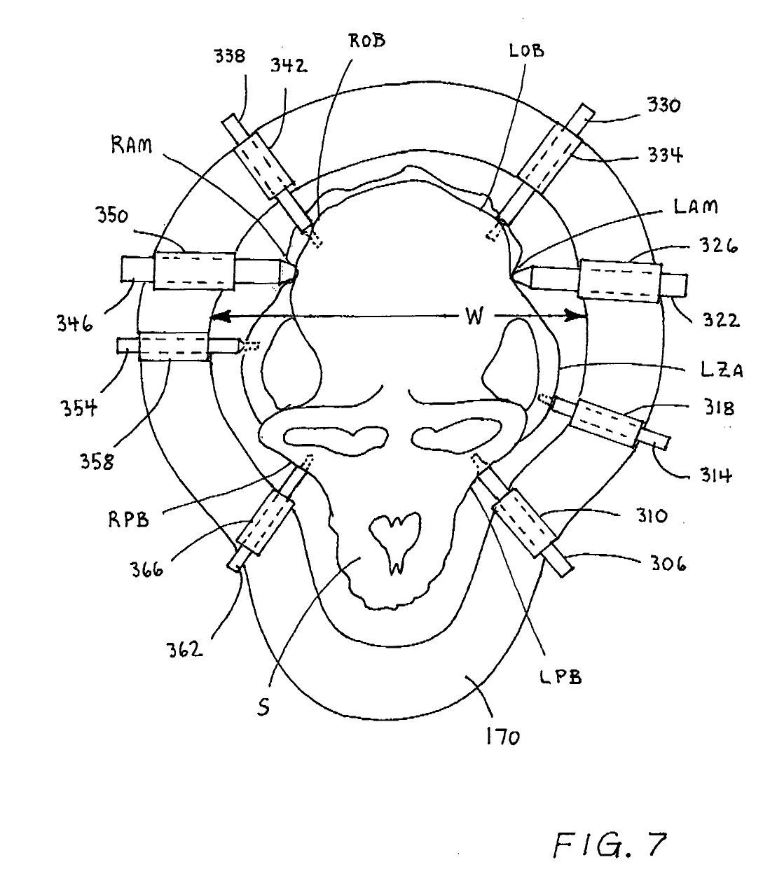

Referring to FIG. 7, the head ring 170 attached to the skull S of a non-human animal is shown from a top view. In different embodiments, different subsets and combinations of types and locations of attachment elements can be used to affix the head ring 170 to the skull S, and FIG. 7 shows some example embodiments. Rod 306 passes through post 310 and attaches to the skull S in the Left Periorbital Bone Region LPB. Rod 314 passes through post 318 and attaches to the skull S in the Left Zygomatic Arch LZA. Ear bar 322 passes through post 326 and is seated in the Left Auditory Meatus LAM. Rod 330 passes through post 334 and attaches to the skull S in the Left Occipital Bone LOB. Rod 338 passes through post 342 and attaches to the skull S in the Right Occipital Bone ROB. Ear bar 346 passes through post 350 and is seated in the Right Auditory Meatus RAM. Rod 354 passes through post 358 and attaches to the skull S in the Right Zygomatic Arch RZA. Rod 362 passes through post 366 and attaches to the skull S in the Right Periorbital Bone Region RPB.

Referring to FIG. 7, the head ring 170 has an inner lateral opening with width W. It can be advantageous to have W be as small as possible and yet still comfortably accommodating the skull size and skin configuration of the animal such as a monkey. This can increase the stability of a placement of ring 170 on skull S because the posts 310, 334, 342, 362, and rods 206, 330 and 362 can be positioned as close to the animal's head and as short a distance from the skull S as is practical. Shorter distances of the fixation element can improve stability and vulnerability of fixation of ring 170 to the skull S. For small monkey skulls, a ring 170 with W less than 8 centimeters (cm) is desirable. For small-to-medium monkey skulls, a ring 170 with W less than 10 cm is desirable. For medium monkey skulls, a ring 170 with W less than 12 cm is desirable. For medium-to-large monkey skulls, a ring 170 with W less than 14 cm is desirable. For skulls of large monkeys, or greater non-human apes, a ring 170 with W less than 16 cm is desirable. For tiny monkeys, such as squirrel monkeys, a ring 170 with W less than 6 cm or 7 cm is desirable.

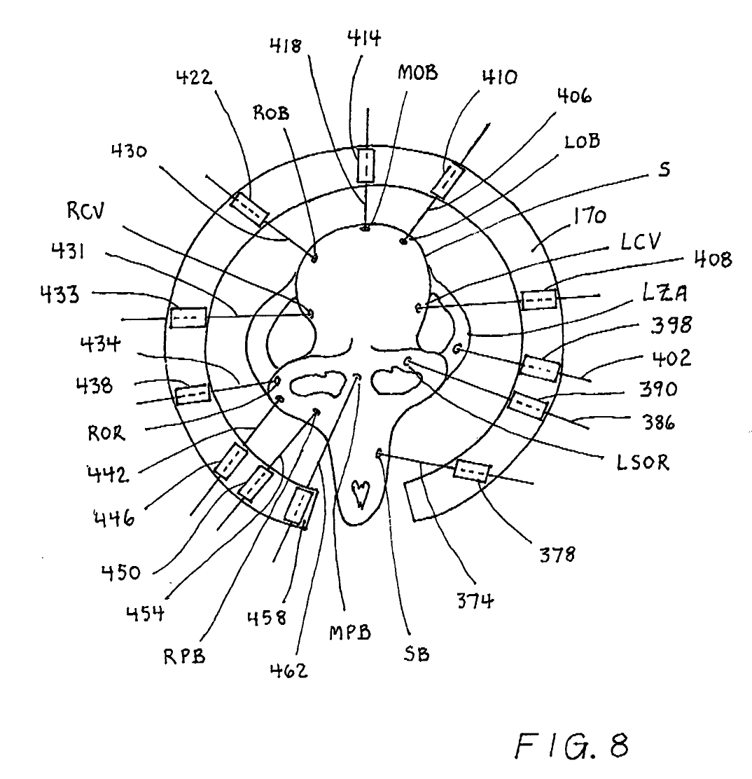

FIG. 8 shows schematically, from the top view, one embodiment of the head ring 170 attached to the skull S of a non-human animal. In different embodiments, different subsets and combinations of types and locations of attachment elements can be used to affix the head ring 170 to the skull S. In different embodiments, each post can be independently adjusted, pre-adjusted and/or fixed in a variety of locations and orientations. Attachment rod 374 associated with post 378 attaches to skull S at a place on the snout bone SB. Attachment rod 386 associated with post 390 attaches to skull S at a place on the left superior orbital ridge LSOR. Attachment rod 402 associated with post 398 attaches to skull S at a place on the left zygomatic arch LZA. Attachment rod 406 associated with post 410 attaches to skull S at a place on the left occipital bone LOB. Attachment rod 418 associated with post 414 attaches to skull S at a place on the medial occipital bone MOB. Attachment rod 430 associated with post 422 attaches to skull S at a place on the right occipital bone ROB. Attachment at the left cranial vault LCV and right cranial vault RCV can be accomplished with post 408 and post 433, respectively, with rods, such as 431 for post 433. Attachment rod 434 associated with post 438 attaches to skull S at a place on the right orbital ridge ROR. Attachment rods 442 and 454, respectively associated with post 446 and 450, attach to the skull at places in the right periorbital region RPB and RPB2, respectively. Attachment rod 462 associated with post 458 attaches to skull S at a place in the medial periorbital bone regions MPB which comprises the bone between the sockets and the ridge of the snout. The posts, such as 378 and 390, can be attached in one example beneath ring 45 and in another example above the ring 458 as viewed in FIG. 8.

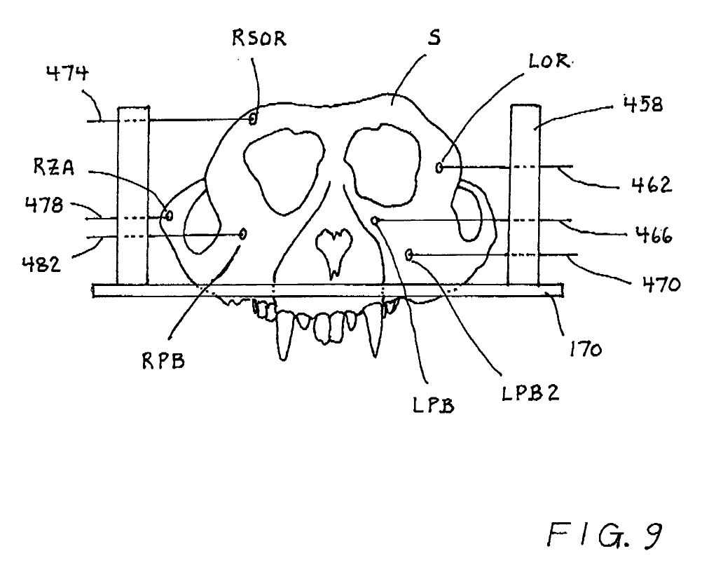

FIG. 9 shows schematically, from the front view, one embodiment of the head ring 170 attached to the skull S of a non-human animal. In different embodiments, the posts associated with attachment rods can have different heights above or below the head ring 170. In different embodiments, the attachment rods can attach to the skull S and/or the post at different or multiple heights above or below the head ring 170. One embodiment of a post 458, for example, can have multiple holes through which rods 462, 466 and 470 can pass. In another embodiment different types of attachment rods can pass through different holes in the same or different posts to suit scientific needs. Attachment rods 462, 466 and 470 are attached to post 458 to attach the head ring 170 to the skull S at a location on the left occipital ridge LOR and at two locations in the left periorbital bone region LPB and LPB2, respectively, and rods 474, 478 and 482 attach the head ring 170 to the skull S at a location on the right superior orbital ridge RSOR, the right zygomatic arch RZA, and the right periorbital bone region RPB, respectively.

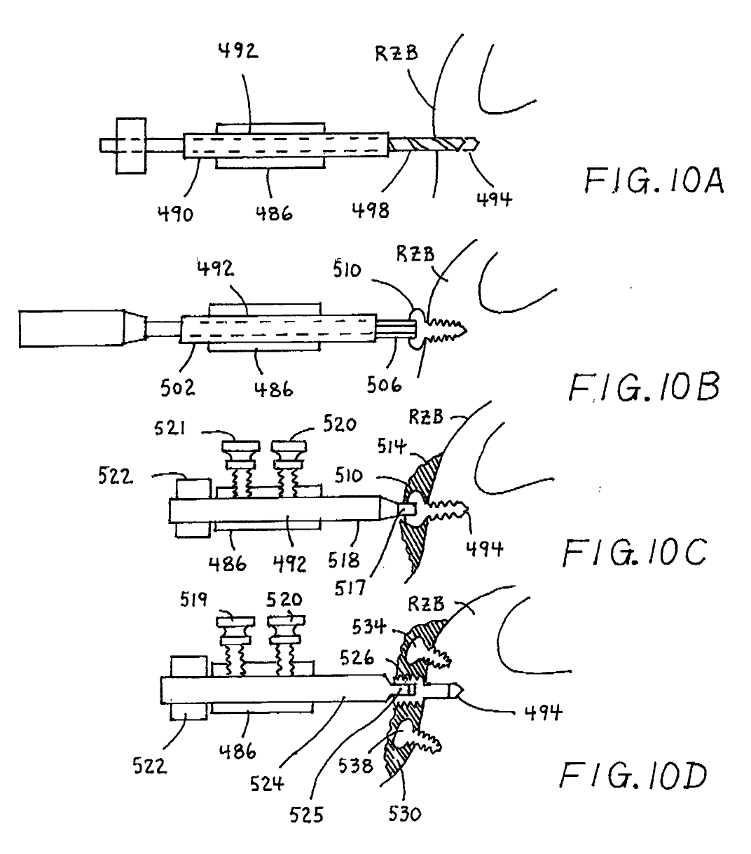

FIGS. 10A, 10B, 10C and 10D show, in cross-section, examples of elements that can be involved in attachment of a stereotactic mechanical reference frame to the right zygomatic bone RZB of skull of a non-human animal. In other embodiments, these elements can be applied to other regions of the skull S, such as the left and right periorbital bone regions, the occipital bone region, the snout bone, the temporal bone, and other bones of the skull S.

Referring to FIG. 10A, a drill 498 passes through bushing 490 seated in or aligned in hole 492 in post 486 that can be one or several posts attached to head ring 170 as, for example, described in the FIGS. 5, 6, 7, 8 or 9. The drill 498 drills a hole 494 in surface of the right zygomatic bone RZB of the skull of a non-human animal. Hole 494 can serve as a pilot hole for a skull screw, a seating hole for an anchoring plug or button, or a positioning burr hole for use in attaching and/or registering a stereotactic mechanical reference frame to skull S. One advantage of passing the drill through the hole in an attachment post 486 is that the drilled hole 494 is aligned with any attachment rod which passes through the same hole in attachment post 486. In another embodiment, hole 494 can be drilled without passing through an attachment post.

Referring to FIG. 10B, a screw driver or wrench 506 passes through bushing 502, seated or guided in post 486 which is attached to head ring 170, to place screw 510 into hole 494 in surface of the right zygomatic bone RZB of the skull of a non-human animal. In another embodiment screw 510 can be screwed into the skull without the use of a pilot hole and/or without passing the screwdriver through post 486. In another embodiment hole 494 can be tapped before screw 510 is placed in it. The tools and steps illustrated in FIG. 10B can be used following the steps in FIG. 10A.

Referring to FIG. 10C, an attachment rod 518 passes through guide hole 492 in post 486 which is attached to head ring 170. The rod 518 has a distal tip 519 that is configured to seat in, lock in, screw into, align in, or attach to a socket or attachment element in screw 510 that is rigidly fixed to the right zygomatic bone RZB of the skull of a non-human animal. Biocompatible glue, such as methyl-methacrylate, indicated by hatched region 514, can adhere or mechanically lock or secure screw 510 to the skull. In another embodiment glue 514 can be omitted. One advantage of glue 514 is that it can provide additional mechanical stability to the attachment of screw 510 to the skull. The end 519 of rod 518 and the head of screw 510 can be configured to attach to each other snugly. Attachment rod 518 can be held rigidly to post 486, for example, by clamping it with screws 519 and/or 520 which screw into threaded holes in attachment post 486 to press against rod 518. Depth stop 522 can be attached to rod 518 and mark or set the position of rod 518 in post 486. One advantage of marking this position is that it can be used for the purpose of a repeat fixation method, in which the head ring 170 associated with post 486 can be repeatedly removed and attached to skull in the same position. By using a depth stop such as 522 that can be clamped on rod 518, and providing such depth stops on the rods in one or several head posts on the ring, a refixation of the rods, such as 518, in the screws, such as 510, can repeatedly attach the ring on the skull in the same position.

One advantage of passing screwdriver 506 through the hole in an attachment post 486 is that the screw 510 can be aligned with attachment rod 518 which later passes through the same hole in attachment post 486. One advantage of pre-drilling hole 494 as a pilot hole for screw 510 is that it helps align screw 510 with attachment rod 518. In one embodiment, attachment rod 518 and/or screw 510 can be configured to attach to each other snuggly when they are aligned in this manner. One advantage of this is that attachment rod 518 and screw 510 attach to each other rigidly. Another advantage of this is that attachment rod 518 and screw 510 can be repeatedly separated and attached in the same position. In another embodiment, the attachment rod 518 can attach directly to bone hole 494. The drill hole 492 and the tip 517 of rod 518 can be configured to match each other and provide desired repositioning of the rod 518 and thus head post 486 and head ring 170 to the skull S.

Referring to FIG. 10D, attachment rod 524 is configured to attach to bushing 526. Bushing 526 is adhered to skull and additional screws 534 and 538, which are themselves attached to skull, by glue which is indicated by the hatched region 530. In other embodiments, one, two, three or more additional screws can be placed in skull and attached to bushing 526 with glue 530. The surface of bushing 526 can be non-smooth to improve adhesion, mechanical locking and/or securing to glue 530. In another embodiment bushing 526 can have a smooth or partially smooth surface. Bushing 526 and/or attachment rod 524 can be configured to attach to each other rigidly. For example, rod 524 can have a shouldered tip 525 that fits snuggly into a hole in bushing 526. Bushing 526 can fit into bone hole 494 by means of a distal tip or shoulder end that fits snuggly into bone hole 494. This has the advantage that bushing 526 is attached to skull more stability. Attachment rod 524 can be held rigidly to post 486, for example, by clamping it with screws 519 and/or 520 which screw into threaded holes in attachment post 486 to press against rod 524. Depth stop 522 can be attached to rod 524 and mark or set the position of rod 524 in post 486. In another embodiment, bushing 526 can be placed without the use of a bone hole. In one embodiment the bushing 526 can be made out of plastic, or some other material which suits scientific needs such as that of causing small or vanishing artifact when scanned by fMRI, MRI, DTI, CT, PET, MEG, EEG, and other scanning methods.

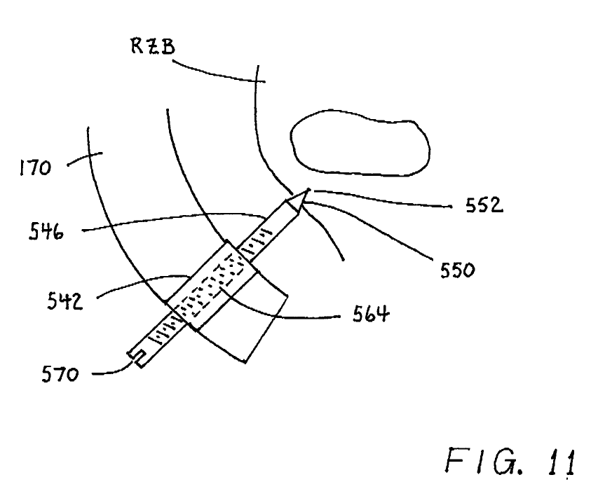

Referring to FIG. 11, from the top view, one embodiment of an attachment rod 546 passes through post 542 attached to head ring 170, and attaches to the right zygomatic bone RZB of the skull of a non-human animal. Attachment rod 546 has a pointed, sharpened end 550 which digs into the skull at point 552. The attachment rod is 546 has a threaded region 558 and passes through a threaded hole 564 in post 542. Attachment rod 546 comprises element 570 which is configured to accept a driver or wrench. In one example, end element 570 comprises a slot which can accept a screwdriver so that rod 446 can be tightened into its skull attachment point 552.

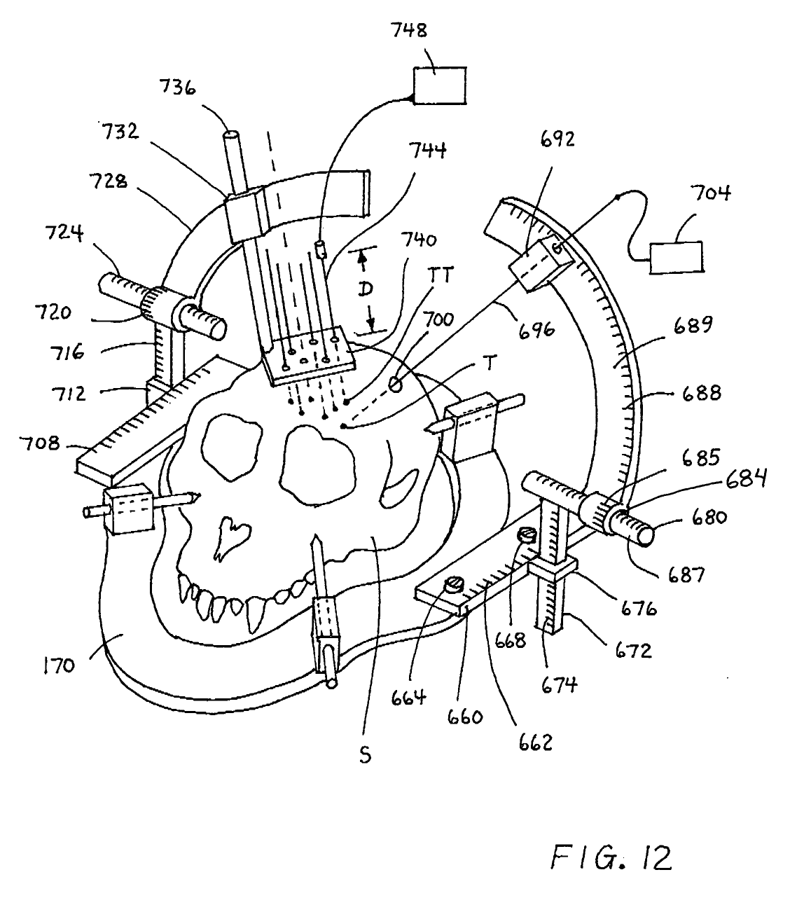

Referring to FIG. 12, a stereotactic guidance system is attached to head ring 170 for the placement of probes into the skull S of a non-human animal. By reference, the CRW system for human stereotaxy includes a stereotactic guidance arc which attaches to a head ring adapted for human heads. This is described in the reference on the CRW stereotactic system described above. One embodiment of a stereotactic guidance system includes a bottom plate and rail 660, which attaches to head ring 170 in a pre-specified, known location with attachment screws 664 and 668. Slider 676 slides along rail 660 and is configured to provide one translational degree of freedom to the stereotactic guidance system, and rail 660 is ruled with position scale markings 662 to show the coordinate along that degree of freedom. Upright 672 slides through slider 676 to provide a different translational degree of freedom to the stereotactic guidance system, and upright 672 is ruled with position scale markings 674 to show the coordinate along that degree of freedom. Horizontal rail 680 is attached to the top of upright 672. Trunion 684 slides along horizontal rail 680 to provide a third and different translational degree of freedom to the stereotactic guidance system, and horizontal rail 680 is ruled to show the coordinate along that degree of freedom. Trunion 684 rotates around horizontal rail 680 to provide a rotational degree of freedom to the stereotactic guidance system. Trunion 684 can be angularly ruled with angle degree markings, such as 685, to show the angular coordinate along that degree of freedom. Arc 688 is attached to trunion 684 and is shaped as a portion of a circular ring or arc. Slider 692 slides along arc 688 to provide another, different rotational degree of freedom to the stereotactic guidance system. Arc 688 can be angularly ruled with angle degree markings, such as 689, to show the angular coordinate along that degree of freedom. Slider 692 can include a probe carrier which holds an electrode, probe, needle or delivery device 696. Slider 692 is configured to allow probe or electrode 696 to be advanced to a specified depth through burr hole 700 in the skull S of a non-human animal to either hit or pass through target T in the head of the non-human animal. The stereotactic guidance arc has three degrees of translational freedom and two degrees of rotational freedom so that probe or electrode 696 can be directed at target T from any direction. The stereotactic guidance arc can be configured to direct probe 696 to target T by using the coordinates of target T relative to the head ring determined using a graphic reference structure and image scanner, such as those shown in FIG. 5. Probe 696 can be associated with system 704 which can have data collection and/or electrical signal generation functions configured to suit scientific needs. For example, probe 696 can have electrode, or agent delivery tubes, or bio-activity sensors which are cooperatively connected to external apparatus 704 by connection 705 to deliver energy, or bio-agents, or drugs, or image tracer agents, such CT or MRI contrast agents, such as Magnesium, for study of the brain anatomy or function at target T or multiple target regions. Probe 696 can be stimulation, recording, or lesioning probe/electrode, and 704 can provide accompanying electronic apparatus, control, computing, or storage. Probe 696 can also be placed using data collected as it is advanced; one advantage of this is that specific types of neurons or tissue can be targeted in the vicinity of a target T.

Referring to FIG. 12, an embodiment of a stereotactic guidance system comprises parts 708, 712, 716, 720, 724, 728, 732, and 736. Rod 736 slides along slider 732 and attaches to probe array plate 740 so that the probe array plate can be advanced toward skull S. Probe array plate 740 can be directed to any location, from any angle using the Stereotactic guidance system; one advantage of this is that the probe array plate can be oriented to facilitate its attachment to skull S. Probe array plate 740 comprises a grid of holes which are configured to hold probes, such as probe/electrode 744. The Stereotactic guidance system can orient the probe plate array 740 so that probes held by it can be directed at pre-specified targets in skull S. For example, probe 744 is advanced to hit target TT. The length D of probe 744 above the probe array plate 740 and the configuration of the stereotactic guidance arc can be used to advance probe 744 to hit target TT. Probes held by array 740 are connected to element 748 which comprises agent injection, bio-agent detection, image enhancement injection, ablation devices or agents, control, signal generation, and/or data collection functions, such as stimulation or recording from the targets associated each probe in the array 740.

In other embodiments, the stereotactic guidance system guides the placement of other types of probes, each with a specific control and/or data collection system, at pre-specified targets in the skull S of a non-human animal. Other types and configurations of probe/electrode carrier can be attached to or reference to the head ring 170 in accordance with the present invention for use in image-scan-guided stereotaxy in non-human animals. For example, articulated arms, robot arms or devices, optically-coupled navigator devices, or magnetically or electro-magnetically tracked navigator devices can be devised by those skilled in the art. Examples of such guidance systems are illustrated in portions of the text on stereotaxy entitled “Stereotactic and Function Neurosurgery”, edited by Philip Gildenberg, M. D., and Ronald R. Tasker, M. D.; 1998; McGraw-Hill Company, which is incorporated in its entirety by reference.

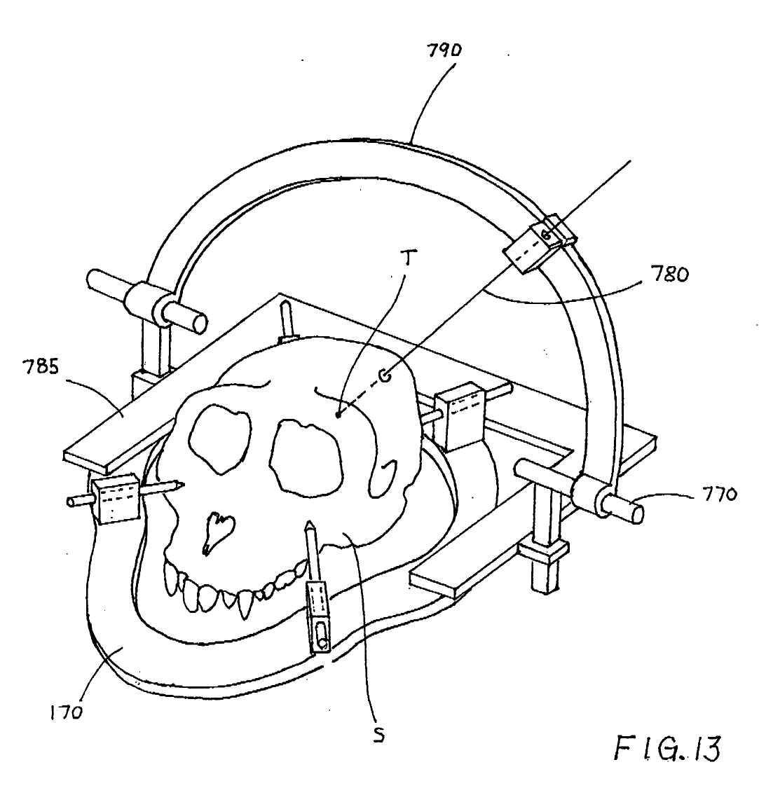

Referring to FIG. 13, another embodiment of a stereotactic guidance system 770 is shown, which can direct a probe 780 at target T at any location in the head and/or skull S of a non-human animal, from any direction. Stereotactic guidance system 770 includes a slider 790 in the shape of a portion of a circular arc which is attached at both of its ends to the rest of the guidance system 770. One advantage of this configuration is that any target location T in the skull S can be achieved by the stereotactic guidance system 770 when attached to the head ring 170 in a single, fixed and known position. The stereotactic guidance system 770 also includes a base 785 which substantially surrounds the skull S. In another embodiment, the base 785 can completely surround the skull S. One advantage of this configuration is mechanical stability.

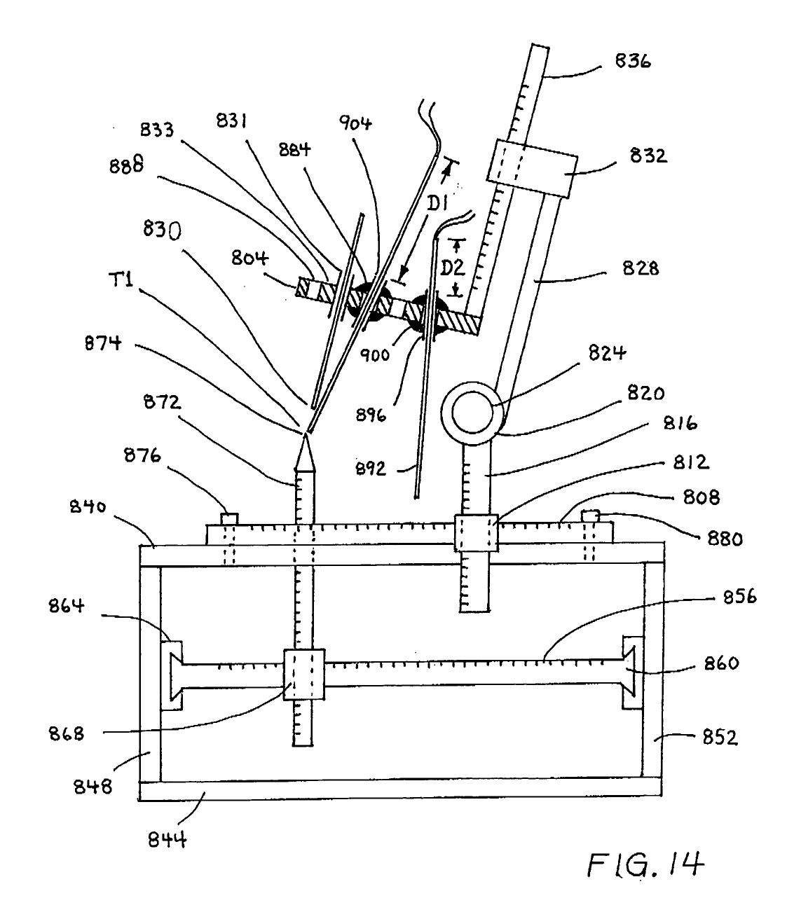

Referring to FIG. 14, shown in partial, sectional view, one embodiment of a stereotactic guidance system can be attached to one embodiment of a phantom base for the purpose of achieving one or more targets in the head of a non-human animal with a probe, needle or delivery device 800 held in an probe array plate 804. One embodiment of a probe array plate 804, shown in cross-section, includes a plate containing one or more holes, such as hole 888, which are configured to hold electrodes, needles, probes, or other delivery or monitoring devices. The embodiment of a stereotactic arc shown in FIG. 14 includes base 808, slider 812, upright 816, trunion 820, horizontal rail 824, arc 828, slider 832, and bar 836. It is analogous to those embodiments shown in FIGS. 12 and 13. The phantom base includes a ring-shaped top plate 840 which can have a shape similar to its corresponding head ring, an example of which is head ring 170. The embodiment of a phantom base shown in FIG. 14 includes a bottom plate 844 and uprights 848 and 852. The phantom base is attached to the stereotactic guidance system in a pre-specified location by attachment screws 876 and 880. The phantom base comprises a pointed rod 872, called the phantom base pointer, whose tip can be moved and fixed at any location T1 achievable by the stereotactic guidance system. Rod 872 slides vertically through slider 868 to provide the pointer 872 with one degree of translational freedom. The rod 872 is ruled to show the coordinate of its tip 874 along that degree of freedom. The slider 868 slides horizontally along rail 856 to provide the pointer 872 with another translational degree of freedom. The rail 856 is ruled to show the coordinate of the tip of rod 872 along that degree of freedom. The rail 856 slides along rails 860 and 864 horizontally in the direction perpendicular to the plane of the page along rails 860 and 864, to give the phantom base one degree of translational freedom. The rails 860 and 864 are ruled to give the coordinate of the tip 874 of rod 872 along that degree of freedom.

The scales on the components of the phantom base, which in this embodiment are pointer 872 and the rails 856, 860 and 864, are configured so that the location of the point tip can be set relative to the coordinate frame of the phantom base. Since the phantom base attaches to the stereotactic guidance system in a known position, the tip of the pointer 872 can be set to a pre-specified location T1 relative to the stereotactic guidance system. Therefore, since the stereotactic guidance system attaches to the head ring 170 in a known position, the tip 874 of the pointer 872 can be set to the 3-D stereotactic target coordinates of a coordinate location of an anatomical target in the skull S of a non-human animal to which the head right 170 is attached. For example, such as anatomical target can be that specified by T in FIG. 1 and FIG. 12. The 3-D coordinates of targets T1 can have been determined from image data of anatomy and index indicia. The stereotactic guidance system can be configured to orient probe array plate 804 so that electrodes, such as 800, 830 and 892, can achieve one or more anatomical target locations, such as T1 and T2, when electrode are placed through the array plate 804 on the phantom base where the coordinates of T1 and T2 have been set. The stereotactic guidance system can also orient the array plate 804 so that it can be attached to the skull S. For example, plate 804 can be separated from slider 836 after probe placement and fixedly attached to the skull S of an animal during the surgical phase of the procedure.

Referring to FIG. 14, in this embodiment, the holes in array plate 804 are cylindrical each with a diameter greater than that of cannula 904. The probe 800 is configured to pass through and be held by the cannula 904; therefore, the probe 800 can pass through array plate 804 in a variety of orientations. The pointer 872 is set to achieve the target location T1. The stereotactic guidance system is configured to orient the array plate 804 so that probes, such as 800, 830 and 892, passing through it can achieve targets, such as T1 and T2. The probe 800 and its cannula 904 are oriented and advanced to achieve target coordinate location T1 so that the cannula 904 passes through a hole in the array plate 804. The cannula 904 can be fixed in this orientation to the array plate 804 by glue, indicated by blacken region 884, which can enter the hole in the array plate. Cannula 896 can be fixed to plate 804 by glue 900 so that electrode 892 achieves target T2. The position of each probe, 800 and 892, can be noted by measuring and/or marking the exposed length, D1 and D2 respectively, when it achieves its target coordinate location, T1 and T2 respectively. The probes 800 and 892 can be removed from the cannula 904 and 896 fixed to the array plate 804, and the phantom base can be detached from the stereotactic guidance system. The stereotactic guidance system can then be attached to the head ring 170 on skull S, as shown in FIG. 12 and FIG. 13. After this, electrodes 800 and 892 can be replaced in their respective cannula 904 and 896 in the same positions, so that the electrodes achieve anatomical targets at the coordinate positions associated with locations T1 and T2. One advantage of this configuration and method is that more than one target coordinate location, such as T1, T2 and others in the vicinity of T1 and T2, can be achieved with high accuracy by multiple probes, such as 800 and 892, held in the array 804.

Referring to FIG. 14, in another embodiment, cannula 831 is guided in hole 833 in plate 804. The probe 830 can be guided in the cannula 831. Close tolerance between the diameters of hole 833, cannula 831, and probe 830 can provide sufficient accuracy to enable probe 830 to approach target position T1 at pointer tip 874 with sufficient accuracy to suit scientific needs with the need to fix probe and cannula with glue as in the samples of probes 800 and 892 just as shown. The coordinates of T1 can be set both on arc slides and phantom vases sliders so that the tip of probe 830 should approach and touch tip 874 at a prescribed advancement distance of 830 inside guide hole 833. This can provide a check of the correctness of the stereotactic coordinate settings associated with target T1 prior to placing the arc on the head ring such as 170 and advancing the probes into the animal's brain to achieve an image-based calculated target position such as T in FIG. 12.

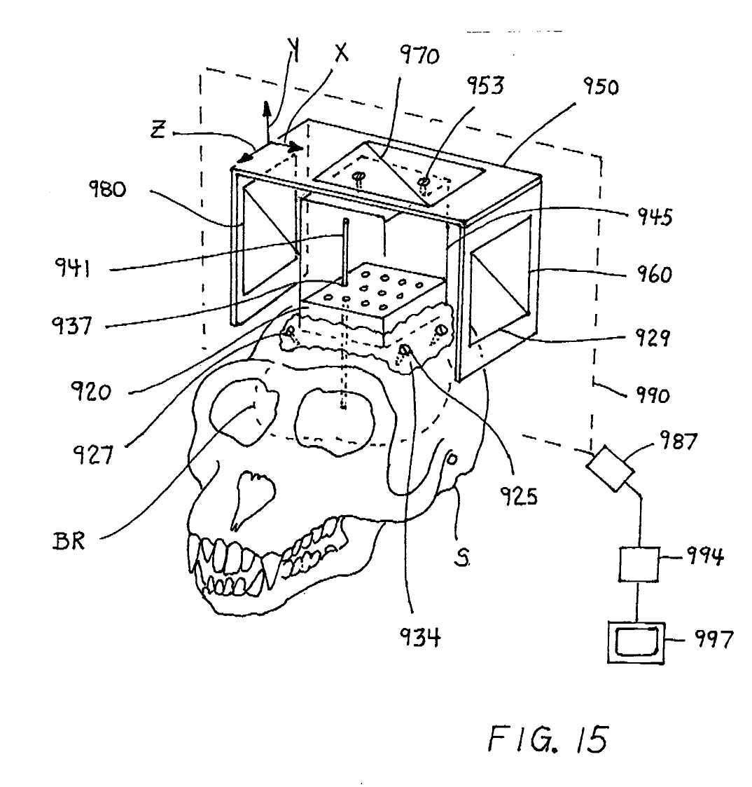

Referring to FIG. 15, a mechanical reference structure includes a probe guide block 920. Block 920 is rigidly attached to the skull S of the non-human animal. The attached includes skull screws such as 925, 927 and 929 which are screwed into the skull bone. Cement or adhesive 934 attaches to the heads of the skull screws 925, 927 and 929, and also attaches to the side edges or bottom of block 920. In one example, block 920 can have a multiplicity of guide holes, such as hole 937, through which a probe shaft such as element 941 can pass and can penetrate into the animal's brain BR. A chamber 945 is attached to block 920, and in one example, can be a box-like structure that can serve to reduce infection around the block 920 and probe 941, and can mechanically protect the probe 941. A graphic reference structure 950 can be rigidly attached to the chamber 945 by screws such as 953. The structure 950 has rod and diagonal graphic indicia structures such as 960, 970 and 980 on the top and left and right sides. The rods and diagonal element of structures 960, 970 and 980 can provide image scan index data similar to the examples described in FIG. 5 and FIG. 6.

An image scanner 987 such as a CT, MRI, PET or other tomographic or volumetric scanner, can produce image data in one or more scan slices, such as in plane 990, which produces index data corresponding to the plane's 990 intersection with the rod and diagonal structures of elements 960, 970 and 980. The image and indicia data can be processed by computer 994 and displayed on display 997. The data from scanner 987, with anatomical and graphic indicia data from structure 950, can enable calculation of target coordinates of anatomy seen in slice 990 with respect to graphic structure 950, that is in coordinates of a three dimensional coordinate system indicated by the axes X, Y, Z defined with respect to the structure 950. Structure 950 can be mechanically fixed in a known position with respect to block 920 so that the position of holes, such as holes 937, can be determined relative to structure 950. In this way, the path of probes such as 941, can be determined relative to localizer 950 and also with respect to the animal's anatomy seen in the image slice data. Multiple scan slices, such as in plane 990, can be gathered to give a volumetric determination of the coordinate relationship of the holes in block 920 and the anatomy such as brain BR. In the this way, the position of anatomy achieved by a probe that is passed through any hole in the block 930 can be calculated as a function of the depth of penetration of a probe through the hole.

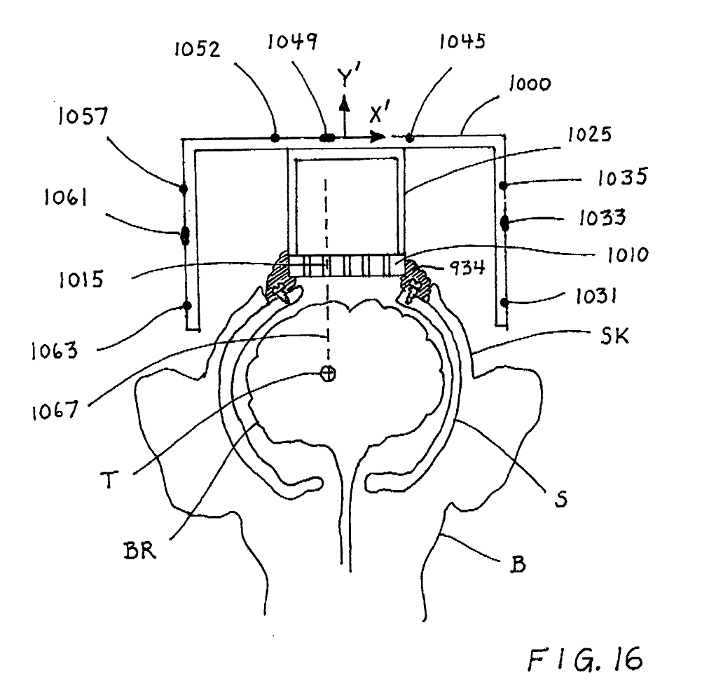

Referring to FIG. 16, a planar sectional view through the anatomy of the body B and the index structure 1000 is shown schematically. In one example, this can represent a tomographic planar image data slice including skull S and skin SK, such as in plane 900, as shown in FIG. 15. The sectional image of block 920 in FIG. 15 is shown in FIG. 16 as element 1010, and the channel hole 937 in FIG. 15 appears as an image channel 1015 in FIG. 16. The slice image of chamber 945 is shown as 1025. The graphic indicia from localizer element 960 appears in FIG. 16 as image spots 1031, 1033 and 1035 for the rods of structures 960; spots 1045, 1049 and 1052 for the rods of structure 970; and spots 1057, 1061 and 1063 for the rods of structure 980. In the image slice plane represented by the image of FIG. 16, there can be an associated two dimensional coordinate system represented by the axes X′,Y′. Each point in the image, such as the centers of the index spots 1031, 1033, 1035, 1045, 1049, 1052, 1057, 1061 and 1063; the hole location 1015 (if it is visible in the scan), and any visible target point, such as point T, can have a definable coordinate location in X′,Y′ space. A transformation can be made between X′,Y′ space and the X,Y,Z coordinate space of the structure 950.

One advantage of the chamber such as 945 and guide block 920 is that they can remain on the animal anatomy for long periods. For example, in brain research, electrodes like probe 941 can be placed in a brain for months or more, and electrical recording can be done for long-term brain experiments. The chamber 950 can protect the electrodes and inhibit infection. An advantage of the localizer 950 attached to chamber 945 is that multiple and long-term repeat image scans can be done during experiments to monitor probe position and/or determine new targets for new probe placement.

Referring to FIG. 15, in one example, the rods and diagonals of structures 960, 970 and 980 can be tubes or channels filled with a medium or solution, such as gadolinium solution, that is visible in an MRI scan. Referring to FIG. 16, if scan 987 is an MRI image plane, the index points such as 1031, 133, 1035, 1045, 1049, 1052, 1057, 1061 and 1063 will appear as dots or ellipses and their X′,Y′ coordinates can be determined. The brain BR of the animal's body B will also be visible in the MRI scan and a target T, accessible by path 1067, can be chosen and it's X′,Y′ coordinates determined. Referring to FIG. 15, the plane of scan 990 can be calculated from the index points, and the position of the hole, such as 937 in grid 920, which produces the best path to target T can be determined. The scan plan 990 can be aligned or non-aligned with the sides or axes of grid 930 and structure 950 to suit the research needs. Many slice images such as 990 can be acquired, and a full three dimensional reconstruction of the position of grid 920 relative to the localizer 950 and relative to the brain BR can be computed by 994 and displayed on element 997. Paths through one or more holes like 937 in grid 920 can be determined, and the position and depths of probes such as 941 can be planned to achieve one or more target positions in the anatomy. In another example, scan 987 can be a plane from CT, PET or another type of tomographic imaging, and the rods and diagonals of structures 960, 970 and 980 can be tubes or channels filled with a medium or solution configured so that they are visible in scan 987.

One advantage for brain research is that targets that are visible in MRI or other image scans can be selected and can be achieved with probes in a calculable, accurate, and proscriptive way using the mechanical reference frame, graphic localizer, and probe guide system show in the FIGS. 1 through 16. In one example, microelectrodes can be accurately placed in desired position in the brain structures based on high resolution MRI or other types of imaging. Confirmation of probe position can be done by repeat scanning using the graphic localizer indicia. Desired probe paths can be selected before insertion by quantitative image scan data. Data from multiple images sources such as MRI, CT, x-ray-, PET and electrical or chemical studies can be combined in one quantitative coordinate basis such as the X,Y,Z coordinate system associated with a structure or a frame for cross-comparison and sequentially timed studies of brain activity.

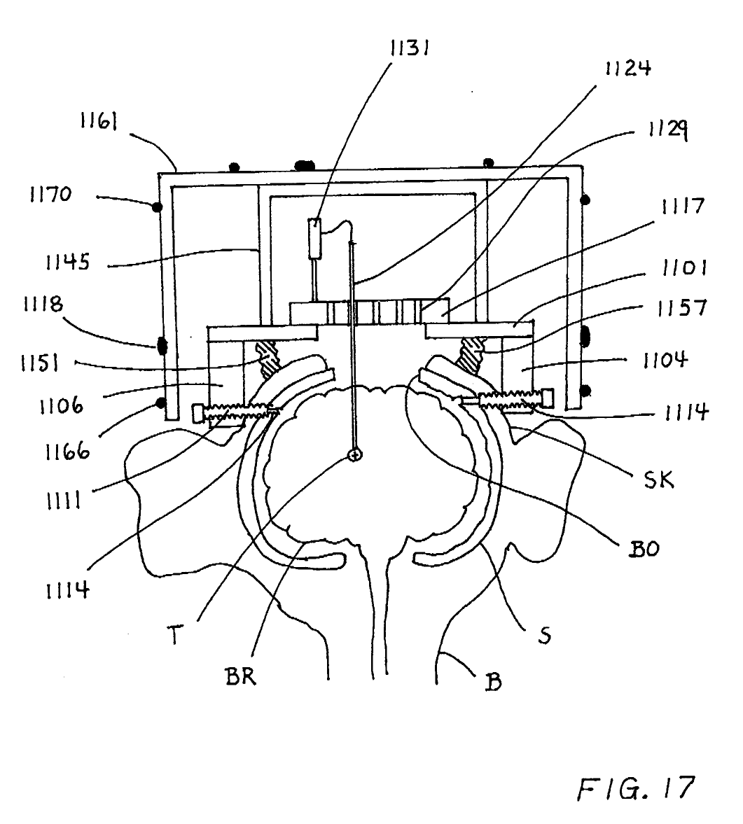

Referring to FIG. 17, a head ring is attached to an animal's skull S in body B. FIG. 17 is a schematic diagram in sectional view though the device and anatomy. There are posts, such as 1104 and 1106, which are below the head ring and skull anchors, such as 1111 and 1114, which pass through the post to anchor the head ring to the skull bone S. A probe guide 1117 is attached to the head ring 1101 and has multiple holes, such as 1117, that can provide access into targets such as T in brain BR by probes such as 1124. In one example, probe 1124 can be an electrical recording probe that is attached to electronic circuit 1131 to stimulate or record brain activity near T. Chamber 1145 encloses one or more probes in carrier 1117. One purpose of the chamber 1145 can be to protect enclosed probes in carrier 117 from being hit. Another purpose of the chamber 1145 can be to act as a barrier against infection. As seal 1151 and 1157 can also prevent dirt and infection from entering the region of the bone and skin opening BO. The graphic reference structure 1161 can be permanently fixed to chamber 1145 or in another case can be attached and then removed from 1145. Index reference structure 1161 has imaging indicia, such as rods and diagonal structures similar to those shown in other embodiments herein. In a tomographic image slice through the localizer 1161, the indicia will be detected as spots such as 1166, 1118 and 1170. From these indicia images, targets such as T can be determined relative to localizer 1161 and relative to frame 1101 and grid 1117, so that desired probe holes, such as hole 1129, and probe paths can be chosen to reach desired target anatomy.

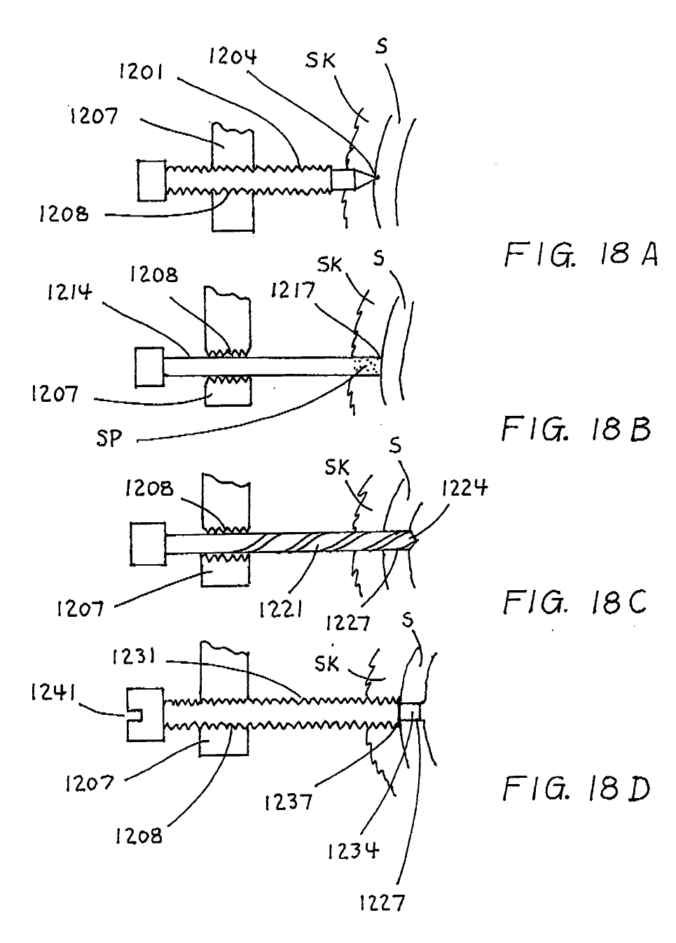

Referring to FIG. 18A, 18B, 18C and 18D, a series of instruments are shown schematically that can be passed through one more head posts, such as 1104 in FIG. 17, to secure a mechanical reference frame to the rigid anatomy of a non-human subject. Referring to FIG. 18A, a portion of a head post 1121 is shown in sectional view having a threaded hold 1208. A screw 1201 is threaded in hole 1208, and it has a pointed tip 1204. Tip 1204 can penetrate soft tissue SK, which in one example can be the scalp. The point 1204 can then dig into the bone anatomy portion S, which can be the skull. In an embodiment involving a head ring such as in FIGS. 3, 4, 5, 6, 7, 8, 9, 12, 13 and 17, multiple sharpened screws like 1201, through multiple head posts, can rigidly fix the head ring to the animal's skull.

Referring to FIG. 18B, a skin punch is shown passing through the hole 1208 of post 1207. In one example, with head ring first secured to the skull with screws such as in FIG. 18A, each screw could be sequentially removed, and a skin punch 1214 can be passed through each post, such as post 1207. The tip 1217 has a sharpened edge so that it can core into or punch a core of skin SP from scalp SK to produce a hole in skin SK down to the bone S.

Referring to FIG. 18C, a drill 1221 can then be passed through hole 1208 after the skin punch core is removed. Drill tip 1224 can make a hole 1227 in skull S.

Referring to FIG. 18D, a shouldered screw 1231 can be threaded into hole 1208 in post 1207 after the hole 1227 has been made as in FIG. 18C. The screw 1231 has a cylindrical tip 1234 which fits into hole 1227, and it has a shouldered edge 1237 which can be driven to the outer edge of bone S to stabilize the penetration of screw 1231 against the bone S. Slot 1241 on the distal end can accommodate a tool to advance screw 1231 into hole 1227 until shoulder 1237 reaches bone S.

For a head ring such as shown in the figures herein, sequential application of the tools shown in FIGS. 18A, 18B, 18C and 18D, one head post after another, can enable the head ring to fix the skull for long periods, as for example, to enable long-term experiments to be performed on an animal subject. An advantage of such as set of fixation instruments and such a method is that the final screw anchors such as 1231 can produce relatively little sustained inward force on the skull bone S to prevent bone erosion and/or necrosis. Also the tip and shoulder provide stable anchoring to the bone S for each post for long term, reliable, fixation position of a mechanical reference frame to the animal's skull.

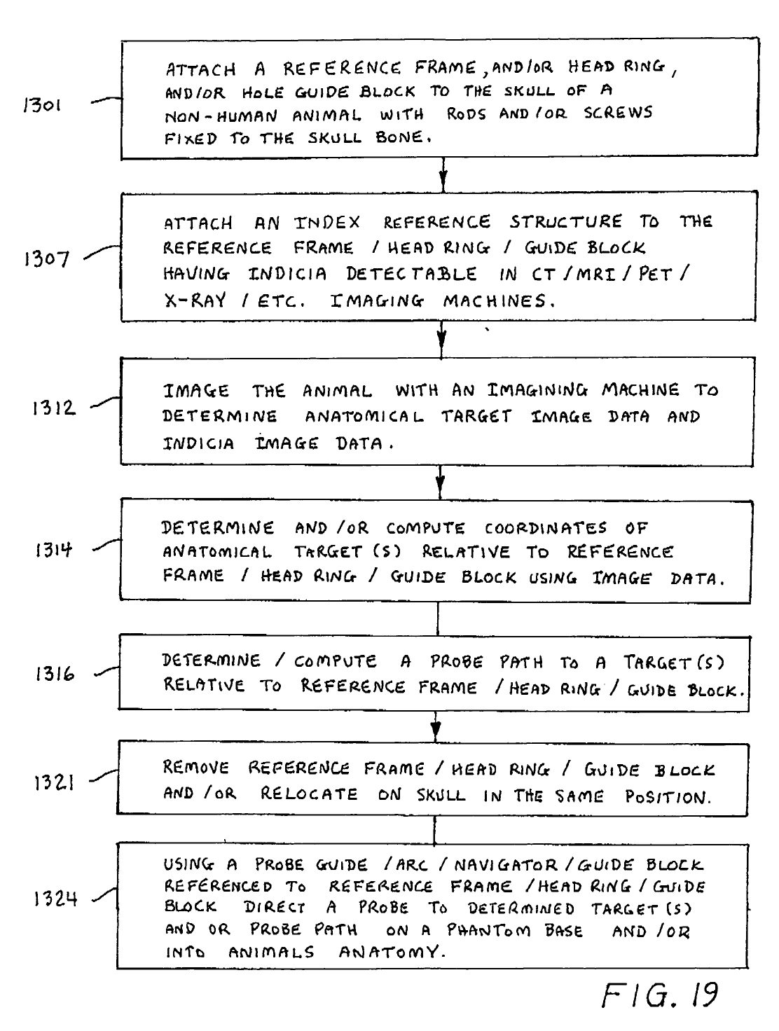

Referring to FIG. 19, a flow chart is shown that illustrates a method of treating the interior of a body at a target location. A mechanical reference frame, such as in one example a head ring, is fixedly attached to the skull of a non-human animal (step 1301). This can be done by attaching or positioning head posts, screws, rods, repeat localizer buttons, or a guide block directly to the skull bone as illustrated in the embodiments of FIGS. 1 through 16. An index reference structure is then attached to the mechanical reference frame in a defined mechanical position (step 1307). The index reference structure can have rod and diagonal elements, spots, dots, markers, spheres, geometric shapes, and/or other graphics indicia that are constructed of a material that can produce detectable indicia data in image data when the index reference structure and the non-human animal's anatomy are imaged by an imaging machine, such as CT, MRI, PET, MEG, EEG, x-ray, infrared, impedance, and other imaging machines. The non-human animal with the mechanical reference frame attached to its skull and the index reference structure attached to the mechanical reference frame, is then imaged by the imaging machine (step 1312). This produces image data corresponding to the non-human animal's anatomy and the indicia of the index reference structure. From the image data and from the geometry of the indicia of the index reference structure, the coordinate position relative to the index reference structure and thus the mechanical reference frame, of anatomical targets or regions detected in the image data can be computed and/or graphically determined (step 1314). A probe path can be computed froma desired direction relative to the animal's anatomy and in the coordinate system of the mechanical reference structure, the index structure, and/or the probe guide (step 1316). The index reference structure can be removed from the mechanical reference frame, and the mechanical reference frame can be removed from the skull of the non-human animal (step 1321). Alternatively, the mechanical reference frame can be kept on the skull of the non-human animal. If the mechanical reference frame is removed, it can be constructed so that it can be repeatedly, and with sufficient accuracy to suit scientific needs, repositioned or refixed on the skull of non-human animal (step 1321). For example, the surgical procedure of placing probes in the non-human animal brain can occur days or months after image data acquisition, and in that case, repeat fixation of the mechanical reference frame to the animal is desirable. A probe guide apparatus such as stereotactic guidance system or digitized navigator can be attached to and/or referenced mechanically to the mechanical reference frame an/or phantom base (step 1324). Alternatively, the mechanical reference frame can have built-in or integral probe paths such as guide holes in a guide block. Probe paths and probe positions can be established on the probe guide to hit target determined in step 1314. If a phantom base is used, correction to probe path and positions can be set up and fixed on a probe carrier or plate to enable a probe or probe array to be established. The probe guide can be fixed to the mechanical reference frame, and the probes can be passed into the brain or spinal region of the non-human animal so that those probes are positioned accurately at targets determined by imaging data. A probe plate, such as a probe array plate which can hold implanted probes, can be fixed to the animal's skull with glue and detached from the probe guide to leave the probes in the brain. Using the localizer illustrated in FIGS. 1, 5 and 6, confirmational lateral and/or frontal x-rays and/or CT, MRI, and/or PET images can be done (also step 1324) to confirm or redetermine the position of probes and their tip positions in the animal's anatomy after surgery or a subsequent times using refixation or the mechanical reference frame at differing time intervals.

Variations of the steps shown in FIG. 19 can be done. For example, steps 1316 and/or 1321 can be eliminated to suit research needs.

The view of these considerations, as well as appreciated by those skilled in the art, implementation and systems should be considered broadly and with reference to the claims set forth below.

1. A method of stereotactic target localization in the body of a non-human subject, comprising:
attaching a mechanical reference frame to the bony structures of the non-human subject, the mechanical reference frame being adapted to support at least one index element and to provide index data when the mechanical reference frame with the at least one index element imaged by an imaging machine to relate the position of the mechanical reference frame to image data from the imaging machine;
imaging the body of the non-human subject and the mechanical reference frame with at least one index element, by the imaging machine to provide image data of anatomical positions in the body and to provide index data from the at least one index element, to provide stereotactic data that relates the positional relationship of the mechanical reference frame and the anatomical positions; and
calculating the positional relationship of a target location in the anatomical positions relative to the mechanical reference frame using the index data and the image data.

2. The method claim 1 wherein the mechanical reference frame is a head frame that is configured to be secured to the skull of a non-human animal by at least one attachment anchor, and wherein attaching includes anchoring the at least one attachment anchor to the skull of the non-human animal.

3. The method of claim 1 further comprising calculating a path to the target locations in relation to the mechanical reference frame based on the stereotactic data.

4. The method of claim 1 further comprising calculating the stereotactic coordinates associated with the image data in the stereotactic coordinate system, wherein the mechanical reference frame has an associated stereotactic coordinate system.

5. The method of claim 3 wherein calculating the path further includes determining a path relative to the probe support so that a probe attached to the probe support can pass to a desired target location determined in the stereotactic data, wherein the mechanical reference frame includes a probe support.

6. A stereotactic system for determining the coordinate position of an anatomical target in the body of non-human subject comprising:
a mechanical reference frame configured to be rigidly attached to the bony anatomy of the non-human subject and to have an associated three-dimensional stereotactic coordinate system; and
an index structure configured to attach to the mechanical reference frame having at least one index element that produces index data in image data when the index element is imaged by an imaging machine, so that when the imaging machine images the non-human subject with the mechanical reference frame and the index structure attached, the coordinate position of the anatomical target in the non-human subject can be determined in the stereotactic coordinate system from the target image data of the anatomical target in the image data.

7. The stereotactic system of claim 6 wherein the mechanical reference frame comprises a head ring structure that is adapted to be rigidly attached to the skull of the non-human subject.

8. The system of claim 6 wherein the index element comprises a tomographic index object that is detectable in at least one scan slice of the index structure by a tomographic imaging scanner to produce the index data.

9. The system of claim 8 wherein the tomographic index object comprises a slice marker element which produces location data in the index data within a single tomographic image slice that define the three-dimensional coordinates of at least three non-collinear points in the stereotactic coordinate system when the index structure is attached to the mechanical reference frame.

10. The system of claim 9 wherein the tomographic index object comprises an MRI index object that indexes data from imaging by an MRI image scanner.

11. The system of claim 9 wherein the slice marker element comprises a diagonal element that is configured to be oriented non-parallel to the plane of the at least one image slice to produce the location data which can be used to determine the orientation of the plane of the tomographic image slice relative to the index structure.

12. The system of claim 11 wherein the diagonal element includes an MRI visible diagonal element that is detectable in the image data from an MRI scanner image.

13. The system of claim 6 further comprising a probe support configured to attach to the mechanical reference frame and support a probe that is aimed at the coordinate position.

14. The system of claim 13 further comprising a phantom base that enables developing a phantom target position on the phantom base at the coordinate position of the anatomical target, whereby the probe support can be attached to the phantom base and a probe path can be developed on the probe support so that when the probe support is attached to the mechanical reference frame, a probe can be passed to the anatomical target in the non-human animal.

15. The system of claim 6 wherein the mechanical reference frame is configured to the size and shape of the skull S of the non-human animal.

16. The system of claim 15 wherein the mechanical reference frame includes a plurality of attachment positions that when selected in combinations rigidly attach to a skull of a non-human subject.
